(19) United States
(12) Patent Application Publication
(10) Pub. No.: US 2003/0004511 A1
(43) Pub. Date: Jan. 2, 2003

(54) Polyaxial pedicle screw system

(76) Inventor: Bret A. Ferree, Cincinnati, OH (US)

Correspondence Address: JERROLD J. LITZINGER, SENTRON MEDICAL, INC., 4445 LAKE FOREST DR., SUITE 600, CINCINNATI, OH, 45242, US

(21) Appl. No.: 10/184,283

(22) Filed: Jun. 27, 2002

Related U.S. Application Data
(60) Provisional application No. 60/301,181, filed on Jun. 27, 2001.

(51) Int. Cl. A61B 17/56

(52) U.S. Cl. 606/61

(57) ABSTRACT

Spinal alignment apparatus including rods, pedicle screws, and hinged connectors for rigidly immobilizing the vertebrae of the spine. The hinged connectors allow angular motion between the pedicle screws and the connectors when the connectors are in an open or unlocked position. The hinged connectors hold the head of the pedicle screw tightly, thus preventing angular motion, when the connector is forced closed. The hinged connector and rod are held closed by a cap or strap over the rod and connector.

### REFERENCE TO RELATED APPLICATION

This application claims benefit from U.S. Provisional Patent Application Serial No. 60/301,181, filed Jun. 27, 2001, the entire contents of which being incorporated herein by reference.

### BACKGROUND OF THE INVENTION

1. Field of the Invention

This invention relates generally to instrumentation and techniques associated with spinal fixation and, in particular, to apparatus and methods facilitating alignment of the spine.

2. Description of the Related Art

Various methods of spinal immobilization have been known and used for many years in the treatment of spinal instability and displacement. As surgical techniques have become more sophisticated, various methods of internal and external fixation have been developed.

External fixation involves at least some portion of the stabilization device which is external to the patient's body. Internal fixation, which is the preferred method, refers to methods of stabilization which are wholly internal to the patient, and commonly involve devices such as screws, plates and rods.

The first successful internal fixation method for surgically treating scoliosis used the Harrington instrumentation system. According to this technique, a rigid rod with hooks at each end is implanted adjacent the concave side of the scoliotic spine. The spine is manually straightened to a desired extent and a distraction rod is used to maintain the correction by exerting vertical forces at each end. The rod commonly has a ratcheted end over which hooks are slidably mounted and locked into place. The Harrington instrumentation system has been successfully used for some time, but because the distraction rod is fixed to the spine in only two places, failure at either end causes the entire system to fail.

An alternative treatment has since evolved which takes advantage of segmented fixation. According to this method, a rod is fixed to the spine at multiple points by means of sublaminar wires, hooks, or pedicle screws.

U.S. Pat. No. 4,611,581 uses pedicle screws that attach to plates on the posterior aspect of the spine. However, the plates can be difficult to insert over pedicle screws angled in several different directions.

U.S. Pat. No. 4,805,602 uses rods that attach to pedicle screws. The device allows angular motion between the pedicle screw and a component that connects the screw to the rod. The motion between the connector component and the screw is restricted by forcing the rod against the pedicle screw. However, the immobilization of the screw is not as rigid as the immobilization of the device taught in '581 patent.

U.S. Pat. No. 4,946,458, like the '602 patent, uses a connector component that allows angular motion of the screw relative to the connector component. The '458 patent teaches the use of nuts on a threaded rod to force two halves of the connector component, contained by a ring, around the screw head, thus immobilizing the screw within the connector component. However, the connector component will fail to hold the screw or the rod if one of the two halves of the connector dissociates from the retaining ring. Furthermore, the device taught in the '458 patent relies on nuts driven on threaded rods. Threaded rods are weaker than rods without threads. Lastly, the use of wrenches on nuts threaded longitudinal rods is cumbersome.

U.S. Pat. No. 5,540,689 uses a shape-memory alloy rod which is deformed at the time of surgery to accommodate the existing curvature of the patient's spine and segmentally affixed using bone clamps, and then heated to effect shape recovery and thereby apply corrective forces to the spine.

While these devices can be adequately used for surgically treating abnormal curvatures of the spine, a need exists for a system that rigidly immobilizes polyaxial pedicle screws within connector components, yet uses rods without threads and has a connector closing mechanism that sits on top of the connector.

### SUMMARY OF THE INVENTION

It is therefore an object of the present invention to provide a device which uses polyaxial pedicle screws which can be securely connected to a solid cylindrical unthreaded rod.

It is a further object to the present invention to provide a spinal alignment system which uses segmented fixation with pedicle screws having connectors through which a cylindrical rod is rigidly affixed at each screw.

It is a still further object of the present invention to provide a pedicle screw system which allows angular motion relative to the rod and connector to achieve maximum holding power without undue stress on the system.

These and other objects of the present invention will be more readily apparent from the description and drawings that follow.

This invention resides in spinal fixation apparatus, including implantable components, instrumentation, and methods of use. In broad and general terms, the preferred embodiment includes pedicle screws that are attached to longitudinal rods by hinged connectors.

The connectors include a hinge feature that permits angular motion of the pedicle screw when open and rigidly immobilizes the pedicle screw when closed. A strap, cap, or clip may be used to hold the hinged connector in position and the rod within the connector.

### BRIEF DESCRIPTION OF THE DRAWINGS

FIG. 1 is a lateral view, partly in cross-section, of a preferred embodiment of the device of the present invention with the rod and pedicle screw locked in position;

FIG. 2 is a lateral view of the device shown in FIG. 1 with the connector in an unlocked position;

FIG. 3 is an exploded view of the device shown in FIG. 1;

FIG. 4 is a lateral view of the cap of the device with the cam in the unlocked position;

FIG. 5 is a lateral view of the cap shown in FIG. 4 with the cam in the locked position;

FIG. 6 is a lateral view, partly in cross-section, of an alternative embodiment of the device of the present invention with the rod and pedicle screw locked in position; and

FIG. 7 is a lateral view of the device shown in FIG. 6 with the connector in an unlocked position.

### DETAILED DESCRIPTION OF THE PREFERRED EMBODIMENTS

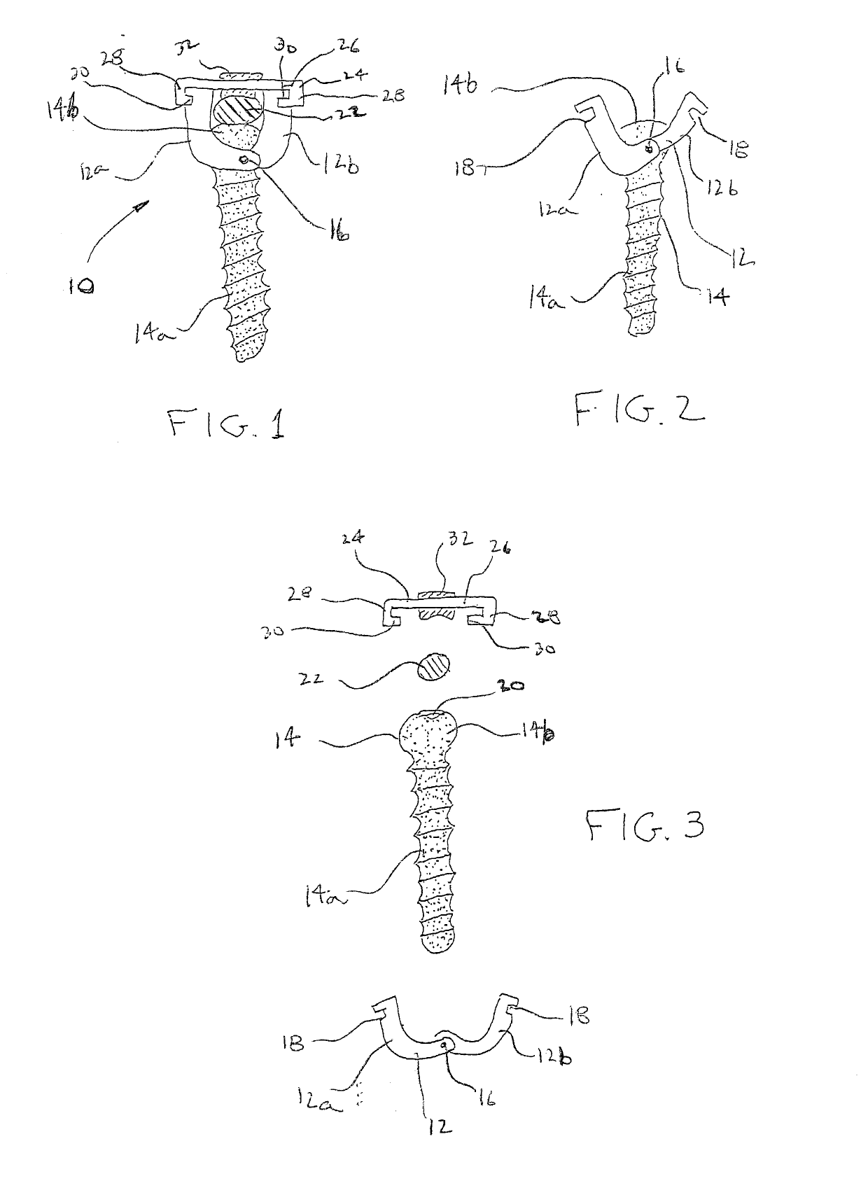

Referring now to FIG. 1, there is shown a locking device for a spinal fixation system, generally indicated at 10, according to the present invention. Devcie 10 includes a hinged connector 12 which is locked around a pedicle screw 14. Hinged connector 12 is formed from two opposing sets of arms 12a, 12b which are each hinged at a pivot 16. Any number of arm sets 12a, 12b more than two can be used. Arm sets 12a, 12b are adjustable from a normal at rest position shown in FIG. 3 to a locking position shown in FIG. 1. In one embodiment, arms 12a, 12b may be biased away from each other in their normal at rest position by a spring or other biasing means known in the art. Each of arms 12a, 12b contain a notched portion 18.

Pedicle screw 14 consists of an exteriorly threaded portion 14a and a spherical head portion 14b. Threaded portion 14a is capable of being threadedly affixed to a vertebra, while head portion 14b is sized such that connector 14 may be inserted over threaded portion 14a of screw 14 until it contacts head portion 14b, allowing rotating motion of connector 12 about head portion 14b. Head portion 14b contains a recess 20, such as a hexagonal recess, to facilitate the installation of pedicle screw 14 into bone by use of a suitable tool.

A cylindrical rod 22 used to couple vertebrae together is positioned above spherical head portion 14b of each pedicle screw 14 which has been inserted into a vertebra at points deemed to be useful to the attending surgeon. Rod 22 may be formed to restore or maintain proper alignment of the spine during the installation procedure of spinal fixation device 10. When locked securely in place by connector 12, neither rod 22 nor screw 14 can move within connector 12.

A cap 24 for locking connector 12 in position about rod 22 and head portion 14b of screw 14 contains a flat planar surface 26 with downwardly depending tabs 28 at each end terminating with inwardly turned edges 30. Edges 30 are sized to fit tightly within notched portions 18 of connector 12 when in the locked position. A cam operated device 32 is rotatably coupled within cap 24 which serves to lock rod 22 in position within connector 12 when device 10 is in use.

FIG. 2 illustrates hinged connector 12 in the unlocked position about screw 14. Screw 14 is free to change angular motion within connector 12 when connector 12 is not held closed or locked by cap 24. Furthermore, cap 24 can be removed, unlocking rod 22 and screw 14, as necessary to reposition rod 22 or screw 14. The distance between screws 14 can be varied by sliding screws 14 and connectors 12 along rod 22 prior to closing locking cap 24. For example, two consecutive screws 14 can be forced apart with an instrument to distract the vertebrae. Head 14b of screw 14 and/or the interior surface of hinged connector 12 can be grooved, sand blasted, or otherwise roughed to increase the friction fit between the head of screw 14 and hinged connector 12.

FIG. 3 shows the insertion sequence of the spinal system. Pedicle screw 14 is inserted through hinged connector 12. Once assembled, each pedicle screw 14 is threaded into a vertebra. Rod 22 is contoured and cut to length to fit the portion of the spine containing pedicle screws 14. Distraction or compression of screws 14 is performed as necessary. A pliers-like instrument (not shown) is used to force the sides of the hinged connector 12 closed. Edges 30 of cap 24 are slid into notches 18 on the hinged connector 12 once connector 12 is closed. The pliers-like instrument can be removed once the cap 24 is partially placed on connector 12. A pliers-like instrument can also be used to force the partially inserted cap 24 completely onto connector 12. Once cap 24 is properly positioned, cam device 32 is rotated 90 degrees, locking rod 22 within connector 12.

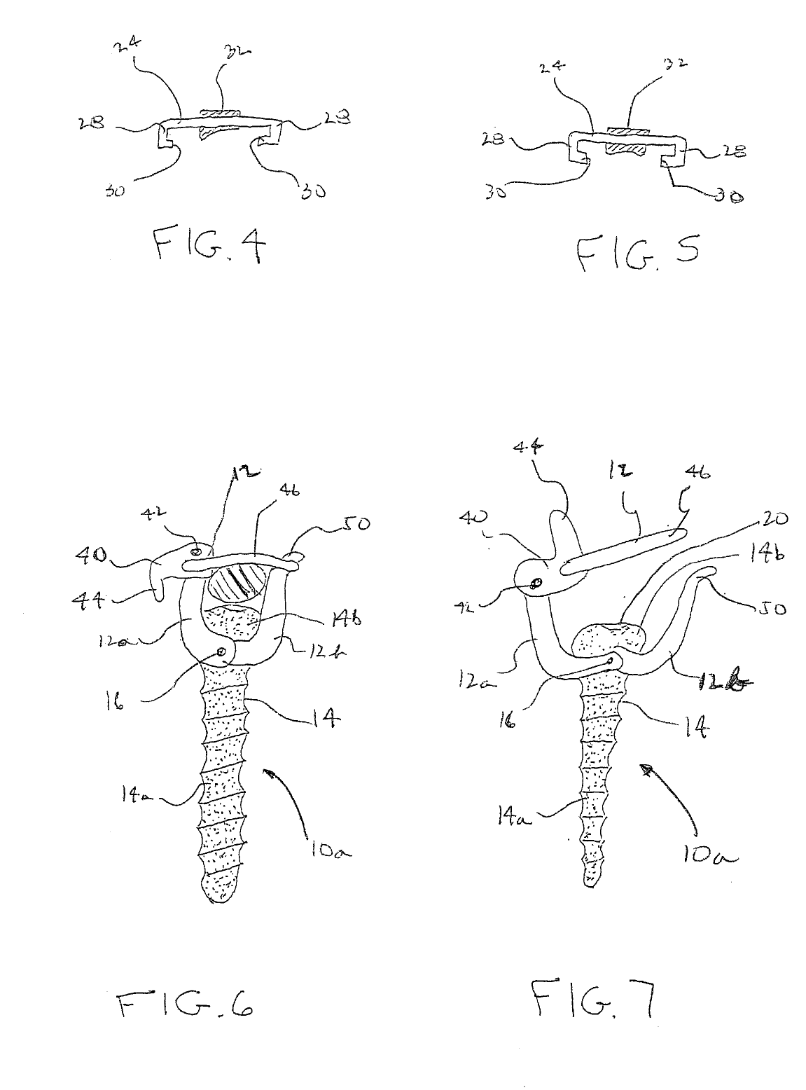

FIG. 4 shows cam device 32 in the unlocked position. Cap 24 can be slid onto connector 12 and over rod 22 when cam device 32 is in the unlocked position. FIG. 5 shows cam 32 in the locked position. Cam 32 fits tight against rod 22 in the locked position, between cam device 32 and head 12a of screw 12.

FIGS. 6 and 7 show an alternative device, generally designated at 10a, for locking rod 22 in position against pedicle screw 14. Referring now to FIG. 6, connector 12 has two opposing sets of arms 12a, 12b which are each hinged at a pivot 16. In this embodiment, 12a, 12b are biased away from each other in their normal at rest by a spring or other biasing means known in the art, as can be seen in FIG. 7. At the end of each arm 12a opposite pivot 16, a lever 40 is rotatably coupled to arm 12a by a pivot 42. Lever 40 terminates in a handle portion 44. Also rotatably coupled to lever 40 is a locking strap 46. At the end of each arm 12b opposite pivot 16, a hook section 50 is formed.

The operation of device 10a can now be described. A screw 14 is inserted through connector 12 up to spherical head 14b, and then section 14a is threaded into a vertebra. Rod 22 is contoured and cut to length to fit the portion of the spine containing pedicle screws 14. Distraction and compression of screws 24 is performed as necessary. In the unlocked position, screw 14 is free to change angular motion within connector 12, and rod 22 can be repositioned.

When device 10a is in the desired position relative to rod 22, lever 40 is rotated about pivot 42 to a point where strap 46 can pass over hook 50 of each arm 12b. After strap 46 is passed over hook 50, handle 44 of lever 40 is rotated about pivot 42 in the opposite direction, causing rod 22 to be captured in position between strap 46, head 14b of pedicle screw 14, and arms 12a, 12b of connector 12. Connector 12 can be unlocked to reposition rod 22 relative to connector 12 or screw 14.

While the present invention has been shown and described in terms of several preferred embodiments thereof, it will be understood that this invention is not limited to these particular embodiments and that changes and modifications may be made without departing from the true spirit and scope of the invention as defined in the appended claims.

What is claimed is:

1. A spinal alignment system, comprising:
at least one pedicle screw having an outer threaded section for securing said screw to a vertebra and a spherical head section coupled to said threaded section;
a connector having at least a first pair of arms and a second pair of arms, said connector capable of being inserted over said threaded section of said pedicle screw adjacent said spherical head section;
a rigid rod capable of contacting said spherical head of each pedicle screw and being held between said first and second pairs of arms of said connector;
and a locking means for engaging said first and second pairs of arms of said connector;
wherein once a desired relationship between said screw and said rod is established, said locking means locks said connector in position to firmly hold said screw and said rod in the desired relationship.

2. The spinal alignment system of claim 1, further comprising adjustment means rotatably coupled to said locking means, having a cam surface used to lock said connector against said rod.

3. The spinal alignment system of claim 1, wherein said locking means comprises a cap.

4. The spinal alignment system of claim 1, wherein said locking means comprises a strap.

5. The spinal alignment system of claim 1, wherein said at least first and second pairs of arms each contain a notch for receiving said locking means.

6. The spinal alignment system of claim 5, wherein said locking means contains a pair of edges which fit into said notches on said arms to hold said arms in a locked position.

7. The spinal alignment system of claim 2, wherein said adjustment means is rotated 90 degrees to lock said connector against said rod.

8. The spinal alignment system of claim 1, further comprising biasing means for forcing said first pair of arms away from said second pair of arms.

9. The spinal alignment system of claim 8, wherein said biasing means comprises a spring.

10. The spinal alignment system of claim 1, wherein said spherical head section of said pedicle screw contains a recess for accommodating a tool for threading said screw into bone.

11. The spinal alignment system of claim 10, wherein said recess has a hexagonal shape.
